(19) United States
(12) Patent Application Publication
(10) Pub. No.: US 2021/0244337 A1
(43) Pub. Date: Aug. 12, 2021

(54) ELECTROCARDIOGRAPH ACQUISITION CIRCUIT, DEVICE, METHOD AND SYSTEM

(71) Applicant: BOE Technology Group Co., Ltd., Beuhubg (CN)

(72) Inventors: Siyang Liang, Beijing (CN); Xun Zhang, Beijing (CN); Hui Du, Beijing (CN)

(21) Appl. No.: 17/049,644

(22) PCT Filed: Apr. 28, 2020
(86) PCT No.: PCT/CN2020/087602

§ 371 Date: Oct. 22, 2020

(30) Foreign Application Priority Data
May 8, 2019 (CN) 201910381002.2

(51) Int. Cl. A61B 5/332; A61B 5/00; A61B 5/024; A61B 5/0245

(52) U.S. Cl. CPC A61B 5/332; A61B 5/681; G16H 40/67; A61B 5/0245; A61B 5/0006; A61B 5/02438

(57) ABSTRACT

An electrocardiograph acquisition circuit, device, method and system are provided. The electrocardiograph acquisition circuit includes: a wireless transmission circuit configured to receive a first electrocardiographic voltage from a second electrocardiograph acquisition device; an electrocardiograph electrode configured to detect an electrical signal on biological skin; an acquisition circuit configured to obtain a second electrocardiographic voltage by acquiring the electrical signal detected by the electrocardiograph electrode; and a control circuit configured to obtain an electrocardiograph index signal by processing the first electrocardiographic voltage received by the wireless transmission circuit and the second electrocardiographic voltage acquired by the acquisition circuit based on a first acquisition time of the first electrocardiographic voltage and a second acquisition time of the second electrocardiographic voltage.

### CROSS-REFERENCE TO RELATED APPLICATION

The present application is a 371 of PCT Application No. PCT/CN2020/087602, filed on Apr. 28, 2020, which claims priority to Chinese Patent Application No. 201910381002.2, filed on May 8, 2019 and entitled “ELECTROCARDIOGRAPH ACQUISITION CIRCUIT, DEVICE, METHOD AND SYSTEM”, the entire contents of which are incorporated herein by reference.

### TECHNICAL FIELD

The present disclosure relates to an electrocardiograph acquisition circuit, device, method, and system.

### BACKGROUND

In daily electrocardiograph monitoring, an electrocardiograph acquisition system may effectively monitor important indexes such as the heart rate and heart rhythm of a patient, and thus is a necessary device for monitoring diseases such as arrhythmia, myocardial ischemia and early coronary heart disease. An electrocardiograph electrode equipped in the electrocardiograph acquisition system acquires electrical signals generated by the heart activity, and the myocardial contraction with each heartbeat will cause a change in a charge on the surface of the biological skin. The electrocardiograph electrode acquires the electrical signal generated by the charge change in this type of activity.

### SUMMARY

Embodiments of the present disclosure provide an electrocardiograph acquisition circuit, device, method and system.

At least one embodiment of the present disclosure provides an electrocardiograph acquisition circuit applicable a first electrocardiograph acquisition device. The electrocardiograph acquisition circuit includes: a wireless transmission circuit, configured to receive a first electrocardiographic voltage from a second electrocardiograph acquisition device; an electrocardiograph electrode, configured to detect an electrical signal on biological skin; an acquisition circuit, configured to obtain a second electrocardiographic voltage by acquiring the electrical signal detected by the electrocardiograph electrode; and a control circuit configured, to obtain an electrocardiograph index signal by processing the first electrocardiographic voltage received by the wireless transmission circuit and the second electrocardiographic voltage acquired by the acquisition circuit based on a first acquisition time of the first electrocardiographic voltage and a second acquisition time of the second electrocardiographic voltage.

Optionally, the first acquisition time and the second acquisition time are times measured from the same initial time.

Optionally, the initial time is a startup time of the first electrocardiograph acquisition device or a startup time of the second electrocardiograph acquisition device.

Optionally, the control circuit is further configured to calculate a first time difference based on the first acquisition time and the second acquisition time when the first acquisition time and the second acquisition time are different, and send the first time difference to the second electrocardiograph acquisition device by the wireless transmission circuit.

Optionally, the wireless transmission circuit is further configured to receive the first acquisition time of the first electrocardiographic voltage from the second electrocardiograph acquisition device.

Optionally, the wireless transmission circuit is further configured to receive a third acquisition time from the second electrocardiograph acquisition device, wherein the third acquisition time is a time measured by taking the startup time of the second acquisition device as an initial time, and

the control circuit is configured to determine whether the first acquisition time and the third acquisition time are the same based on a second time difference between the startup time of the first electrocardiograph acquisition device and the startup time of the second electrocardiograph acquisition device, the second acquisition time and the first acquisition time.

Optionally, the wireless transmission circuit is further configured to receive an instruction from the second electrocardiograph acquisition device at the startup time; and

the control circuit is configured to determine the second time difference between the startup time of the first electrocardiograph acquisition device and the startup time of the second electrocardiograph acquisition device based on the instruction.

Optionally, the control circuit is further configured to obtain the electrocardiograph index signal by calculating a differential signal between the second electrocardiographic voltage and the first electrocardiographic voltage when the first acquisition time and the second acquisition time are the same.

Optionally, the control circuit is further configured to obtain a common mode signal by averaging the second electrocardiographic voltage and the first electrocardiographic voltage, and send the common mode signal to a device with a drive circuit by the wireless transmission circuit.

At least one embodiment of the present disclosure provides an electrocardiograph acquisition circuit applicable to a second electrocardiograph acquisition device. The electrocardiograph acquisition circuit includes:

an electrocardiograph electrode, configured to detect an electrical signal on biological skin;

an acquisition circuit, configured to obtain a first electrocardiographic voltage by acquiring the electrical signal detected by the electrocardiograph electrode; and

a wireless transmission circuit, configured to send the first electrocardiographic voltage to a first electrocardiograph acquisition device.

Optionally, the wireless transmission circuit is further configured to send a first acquisition time of the first electrocardiographic voltage, wherein the first acquisition time and a second acquisition time are times measured from the same initial time, and the second acquisition time is a time when the first electrocardiograph acquisition device acquires a second electrocardiographic voltage; or

the wireless transmission circuit is further configured to send a third acquisition time of the first electrocardiographic voltage, wherein the third acquisition time is a time measured by taking a startup time of the second acquisition device as an initial time.

Optionally, the wireless transmission circuit is further configured to receive a first time difference from the second electrocardiograph acquisition device; and

a control circuit is configured to output a first clock signal, wherein the phase of the first clock signal based on the first time difference received by the wireless transmission circuit is adjusted to enable the first clock signal to be synchronized with a second clock signal, wherein the second clock signal is a clock signal of the second electrocardiograph acquisition device, and the first clock signal has the same frequency as the second clock signal.

At least one embodiment of the present disclosure provides an electrocardiograph acquisition device. The electrocardiograph acquisition device includes: an electrocardiograph acquisition circuit, a housing, and a wristband connected to the housing, wherein the electrocardiograph acquisition circuit is the electrocardiograph acquisition circuit applicable to the first electrocardiograph acquisition device as mentioned above, or the electrocardiograph acquisition circuit applicable to the second electrocardiograph acquisition device as mentioned above;

the electrocardiograph electrode is embedded on a surface of the housing; and

the wireless transmission circuit, the acquisition circuit, and the control circuit are disposed in the housing.

At least one embodiment of the present disclosure provides an electrocardiograph acquisition system. The electrocardiograph acquisition system includes: a master device and a slave device, wherein the master device is a first electrocardiograph acquisition device including the electrocardiograph acquisition circuit applicable to the first electrocardiograph acquisition device as mentioned above, and the slave device is a second electrocardiograph acquisition device including the electrocardiograph acquisition circuit applicable to the second electrocardiograph acquisition device as mentioned above.

Optionally, the electrocardiograph acquisition system includes two slave devices, wherein the master device and the two slave devices are placed on two hands and one leg of a human body respectively.

Optionally, the electrocardiograph acquisition system further includes a device with a drive circuit.

Optionally, the device with the drive circuit includes:

a wireless transmission circuit, configured to receive a common mode signal from the master device;

a drive circuit, configured to generate a current signal by processing the common mode signal received by the wireless transmission circuit; and

an electrocardiograph electrode, configured to output the current signal generated by the drive circuit to the biological skin.

At least one embodiment of the present disclosure provides an electrocardiograph acquisition method applicable to any of the above electrocardiograph acquisition circuits. The method includes:

receiving a first electrocardiographic voltage from a second electrocardiograph acquisition device by the wireless transmission circuit;

obtaining a second electrocardiographic voltage by acquiring an electrical signal detected by the electrocardiograph electrode from the biological skin by the acquisition circuit; and

obtaining an electrocardiograph index signal by processing the first electrocardiographic voltage received by the wireless transmission circuit and the second electrocardiographic voltage acquired by the acquisition circuit based on a first acquisition time of the first electrocardiographic voltage and a second acquisition time of the second electrocardiographic voltage.

At least one embodiment of the present disclosure provides an electrocardiograph acquisition method applicable to the above electrocardiograph acquisition circuit. The method includes:

obtaining a first electrocardiographic voltage by acquiring an electrical signal detected by the electrocardiograph electrode from the biological skin by the acquisition circuit; and

sending the first electrocardiographic voltage to a first electrocardiograph acquisition device.

### BRIEF DESCRIPTION OF DRAWINGS

FIG. 1 is a structural block diagram of an electrocardiograph acquisition circuit according to an embodiment of the present disclosure;

FIG. 2 is a structural block diagram of another electrocardiograph acquisition circuit according to an embodiment of the present disclosure;

FIG. 3 is a structural diagram of an acquisition circuit according to an embodiment of the present disclosure;

FIG. 4 is a structural block diagram of a control circuit according to an embodiment of the present disclosure;

FIG. 5 is a circuit diagram of the control circuit according to the embodiment of the present disclosure;

FIG. 6 and FIG. 7 are structural diagrams of an electrocardiograph acquisition terminal in two opposite directions respectively according to an embodiment of the present disclosure;

FIG. 8 is a structural diagram of an electrocardiograph acquisition system according to an embodiment of the present disclosure;

FIG. 9 is a diagram showing the position of the electrocardiograph acquisition system shown in FIG. 8;

FIG. 10 is a structural diagram of a device with a drive circuit according to an embodiment of the present disclosure;

FIG. 11 is a flow chart of an electrocardiograph acquisition method according to an embodiment of the present disclosure; and

FIG. 12 is a flow chart of an electrocardiograph acquisition method according to an embodiment of the present disclosure.

### DESCRIPTION OF EMBODIMENTS

For clearer descriptions of the objects, technical solutions and advantages of the present disclosure, embodiments of the present disclosure will be described in detail below in combination with the accompanying drawings.

The electrocardiograph acquisition system usually includes a plurality of electrocardiograph electrodes that need to be set in the limbs, chest, abdomen and the like of a human body to detect charge changes generated at these positions during the myocardial contraction with each heartbeat. An electrocardiograph electrode system also includes a terminal. The electrical signals acquired by various electrocardiograph electrodes need to be transmitted to the terminal by a transmission line, and the terminal will finally process the electrical signals generated by these electrocardiograph electrodes.

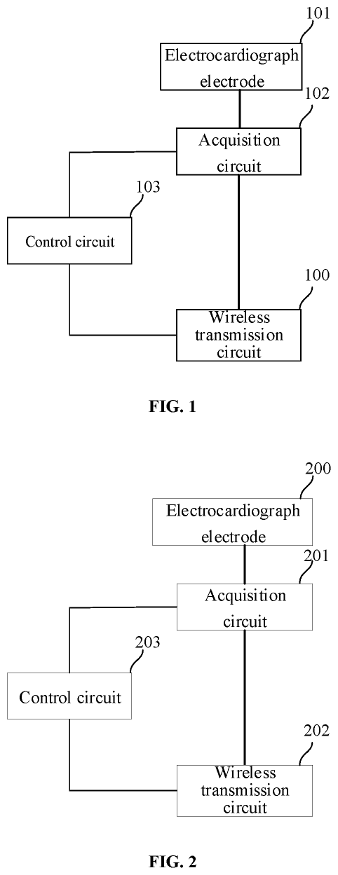

FIG. 1 is a structural block diagram of an electrocardiograph (electrocardiograph) acquisition circuit according to an embodiment of the present disclosure. With reference to FIG. 1, the electrocardiograph acquisition circuit is applicable to a first electrocardiograph acquisition device (i.e., a master device), and includes: a wireless transmission circuit 100, an electrocardiograph electrode 101, an acquisition circuit 102 and a control circuit 103.

The wireless transmission circuit 100 is configured to receive a first electrocardiographic voltage from a second electrocardiograph acquisition device.

The electrocardiograph electrode 101 is configured to detect an electrical signal on biological skin.

The acquisition circuit 102 is configured to obtain a second electrocardiographic voltage by acquiring the electrical signal detected by the electrocardiograph electrode 101.

The control circuit 103 is configured to obtain an electrocardiograph index signal by processing the first electrocardiographic voltage received by the wireless transmission circuit 100 and the second electrocardiographic voltage acquired by the acquisition circuit 102 based on a first acquisition time of the first electrocardiographic voltage and a second acquisition time of the second electrocardiographic voltage.

In this electrocardiograph acquisition circuit, the signal is detected by the electrocardiograph electrode, and then the electrocardiographic voltage is acquired and processed by the acquisition circuit and the control circuit. In addition, the electrocardiographic voltage is received from the second electrocardiograph acquisition device by the wireless transmission circuit. Since the signal between the electrocardiograph acquisition devices is transmitted wirelessly, there is no need to dispose a connection line on the electrocardiograph acquisition device. Thus, the comfortability for a user to wear the electrocardiograph acquisition device is improved and hence the user can move freely.

The biological skin includes but is not limited to human skin, or other animal skin.

In the embodiment of the present disclosure, the first acquisition time and the second acquisition time are times measured from the same initial time. As the acquisition times of the two electrocardiographic voltages are based on the same initial time, whether the two electrocardiographic voltages are synchronized is determined conveniently, thereby facilitating synchronization processing.

Exemplarily, the initial time is a startup time of the first electrocardiograph acquisition device or a startup time of the second electrocardiograph acquisition device. For example, the startup time of the first electrocardiograph acquisition device may be used as the initial time.

In the embodiment of the present disclosure, the control circuit 103 is further configured to calculate a first time difference based on the first acquisition time and the second acquisition time when the first acquisition time and the second acquisition time are different, and send the first time difference to the second electrocardiograph acquisition device by the wireless transmission circuit 100. As a result, the second electrocardiograph acquisition device may adjust the acquisition time and thus the first acquisition time and the second acquisition time are synchronized.

In some embodiments, the wireless transmission circuit 100 is further configured to receive the first acquisition time of the first electrocardiographic voltage from the second electrocardiograph acquisition device. For example, in the case that the startup time of the first electrocardiograph acquisition device is used as the initial time, the first electrocardiograph acquisition device may firstly send a time difference between the startup time of the first electrocardiograph acquisition device and the startup time of the second electrocardiograph acquisition device to the second electrocardiograph acquisition device, such that the second electrocardiograph acquisition device may determine the first acquisition time of the first electrocardiographic voltage by taking the startup time of the first electrocardiograph acquisition device as the initial time. For another example, in the case that the startup time of the second electrocardiograph acquisition device is used as the initial time, the second electrocardiograph acquisition device directly records the acquisition time and then sends the acquisition time to the first electrocardiograph acquisition device.

In some embodiments, the wireless transmission circuit 100 is further configured to receive a third acquisition time of the first electrocardiographic voltage from the second electrocardiograph acquisition device. The third acquisition time and the second acquisition time are times measured from different initial times. For example, in the case that the startup time of the second electrocardiograph acquisition device is used as the initial time, the second electrocardiograph acquisition device directly records the acquisition time and then sends the acquisition time to the first electrocardiograph acquisition device.

In the embodiment of the present disclosure, when the wireless transmission circuit 100 receives the third acquisition time from the second electrocardiograph acquisition device, the control circuit 103 is configured to determine whether the first acquisition time and the second acquisition time are the same based on a second time difference between the startup time of the first electrocardiograph acquisition device and the startup time of the second electrocardiograph acquisition device, the second acquisition time and the third acquisition time.

Optionally, the wireless transmission circuit 100 is further configured to receive an instruction from the second electrocardiograph acquisition device at the startup time; and

the control circuit 103 is configured to determine the second time difference between the startup time of the first electrocardiograph acquisition device and the startup time of the second electrocardiograph acquisition device based on the instruction.

In the embodiment of the present disclosure, the control circuit 103 is further configured to obtain the electrocardiograph index signal by calculating a differential signal between the second electrocardiographic voltage and the first electrocardiographic voltage when the first acquisition time and the second acquisition time are the same.

In the embodiment of the present disclosure, the control circuit 103 is further configured to obtain a common mode signal by averaging the second electrocardiographic voltage and the first electrocardiographic voltage, and send the common mode signal to a device with a drive circuit by the wireless transmission circuit 100.

FIG. 2 is a structural block diagram of another electrocardiograph acquisition circuit according to an embodiment of the present disclosure. With reference to FIG. 2, the electrocardiograph acquisition circuit is applicable to a second electrocardiograph acquisition device (i.e., a slave device), and includes: an electrocardiograph electrode 200, an acquisition circuit 201, a wireless transmission circuit 202 and a control circuit 203.

The electrocardiograph electrode 200 is configured to detect an electrical signal on biological skin.

The acquisition circuit 201 is configured to obtain a first electrocardiographic voltage by acquiring the electrical signal detected by the electrocardiograph electrode 200.

The wireless transmission circuit 202 is configured to send the first electrocardiographic voltage to a first electrocardiograph acquisition device.

In the embodiment of the present disclosure, the wireless transmission circuit 202 is further configured to send a first acquisition time of the first electrocardiographic voltage or a third acquisition time of the first electrocardiographic voltage. The first acquisition time and a second acquisition time are times measured from the same initial time, the third acquisition time and the second acquisition time are times measured from different initial times, and the second acquisition time is a time when the first electrocardiograph acquisition device acquires a second electrocardiographic voltage.

Herein, regarding whether the second electrocardiograph acquisition device sends the first acquisition time or the third acquisition time, reference may be made to the above description about whether the wireless transmission circuit 100 receives the first acquisition time or the third acquisition time.

In the embodiment of the present disclosure, the wireless transmission circuit 202 is further configured to receive a first time difference from the first electrocardiograph acquisition device.

The control circuit 203 is further configured to output a first clock signal. The phase of the first clock signal based on the first time difference received by the wireless transmission circuit 202 is adjust to enable the first clock signal to be synchronized with a second clock signal. The second clock signal is a clock signal of the first electrocardiograph acquisition device, and the first clock signal has the same frequency as the second clock signal.

The following exemplarily describes the solution provided by the present disclosure with an example that the startup time of the first electrocardiograph acquisition device is taken as the initial time and the second electrocardiograph acquisition device sends the first electrocardiographic voltage and the third acquisition device of the first electrocardiographic voltage to the first electrocardiograph acquisition device at the same time.

In the first electrocardiograph acquisition device, the wireless transmission circuit 100 is configured to receive a first electrocardiographic signal from the second electrocardiograph acquisition device. The first electrocardiographic signal includes the first electrocardiographic voltage and the third acquisition time of the first electrocardiographic voltage. The electrocardiograph electrode 101 is configured to detect the electrical signal on biological skin. The acquisition circuit 102 is configured to obtain the second electrocardiographic voltage by acquiring the electrical signal detected by the electrocardiograph electrode 101. The control circuit 103 is configured to generate a second electrocardiographic signal obtained by the acquisition circuit 102 that is based on the second electrocardiographic voltage. The second electrocardiographic signal includes the second electrocardiographic voltage and the second acquisition time of the second electrocardiographic voltage. The first acquisition time is determined based on the third acquisition time. The electrocardiograph index signal is obtained by processing the first electrocardiographic signal received by the wireless transmission circuit 100 and the second electrocardiographic signal generated by the control circuit 103 based on the first acquisition time and the second acquisition time.

The electrocardiograph electrode 101 may be a round metal electrode sheet through which the electrical signal on the biological skin is acquired.

The electrocardiograph index signal is configured to indicate an electrocardiograph index of a user, such as the user's heart rate.

The wireless transmission circuit carries the acquisition time of the electrocardiographic voltage when transmitting the electrocardiographic voltage, such that the electrocardiograph acquisition circuit may determine the electrocardiographic voltages acquired at the same time according to the acquisition time, thereby ensuring the synchronization of signals of various electrocardiograph acquisition devices and avoiding reduction in signal acquisition quality caused by the wireless transmission.

The electrocardiograph acquisition circuit shown in FIG. 1 is an electrocardiograph acquisition circuit of a master device in an electrocardiograph acquisition system, and the second electrocardiograph acquisition device is a slave device in the electrocardiograph acquisition system. The master device is wirelessly connected to the slave device, and the master device calculates the electrocardiograph index by its own detection and data reported from the slave device.

Exemplarily, the wireless transmission circuit 100 of the electrocardiograph acquisition circuit may use technologies such as Bluetooth, Wi-Fi for wireless transmission.

FIG. 3 is a structural diagram of the acquisition circuit according to the embodiment of the present disclosure. With reference to FIG. 3, the electrocardiograph electrode 101 is disposed on a human body 10.

The acquisition circuit 102 includes a front-end circuit 121 and an analog-to-digital converter 122. The front-end circuit 121 is configured to convert a current signal detected by the electrocardiograph electrode 101 into an analog voltage signal. The analog-to-digital converter 122 is configured to obtain a digital voltage signal (i.e., the above second electrocardiographic voltage) by periodically sampling the analog voltage signal output by the front-end circuit 121 under the control of the clock signal.

In order to ensure the synchronization of electrocardiographic signals acquired by various electrocardiograph acquisition devices, the clock signals of the acquisition circuits of the various electrocardiograph acquisition devices need to adopt a uniform frequency.

The clock signal may be provided by the above control circuit 103. Therefore, the control circuit 103 may determine the second electrocardiographic signal according to the second electrocardiographic voltage and the clock signal.

For example, when the clock signal is a square wave signal, a rising edge of the square wave signal may be used to control the acquisition circuit 102 to acquire the second electrocardiographic voltage. When one second electrocardiographic voltage is acquired each time, a time corresponding to the rising edge when the second electrocardiographic voltage is acquired is taken as the second acquisition time, and the second electrocardiographic signal is obtained by combining the second electrocardiographic voltage and the second acquisition time.

With reference to FIG. 3, the front-end circuit 121 includes a resistor r1, a resistor r2, a resistor r3, a capacitor c1 and a capacitor c2. The resistor r1 and the resistor r2 are sequentially connected in series between the electrocardiograph electrode 101 and the analog-to-digital converter 122. One end of the capacitor c1 is connected between the resistor r1 and the resistor r2, and the other end of the capacitor c1 is grounded. The capacitor c2 is connected in parallel with two ends of the resistor r2. One end of the resistor r3 is connected between the resistor r2 and the analog-to-digital converter 122, and the other end of the resistor r3 is grounded. The front-end circuit 121 of this circuit structure can achieve current-to-voltage conversion.

In the embodiment of the present disclosure, when obtaining the first electrocardiographic signal and the second electrocardiographic signal, the control circuit 103 needs to firstly determine whether the first acquisition time of the first electrocardiographic voltage and the second acquisition time of the second electrocardiographic voltage are the same, and then performs subsequent processing to ensure the synchronization of the processed electrocardiographic signals.

When the first electrocardiograph acquisition device is taken as the master device, the electrocardiograph acquisition circuit of the first electrocardiograph acquisition device may calculate a time difference between the acquisition time of each device and the acquisition time of the master device based on the time when each electrocardiograph acquisition device acquires the electrocardiographic voltage, and send the time difference to each second electrocardiograph acquisition device. Thus, these electrocardiograph acquisition devices may adjust the acquisition times according to the time differences, thereby realizing the synchronization of the electrocardiographic voltages acquired by various electrocardiograph acquisition devices.

Since a plurality of slave devices may be configured, the control circuit 103 may calculate the first time difference for each of the second electrocardiograph acquisition devices and send the first time difference to the corresponding second electrocardiograph acquisition device. The time differences calculated by the master device for different electrocardiograph acquisition devices may be the same or different.

In order to facilitate distinction of the ownership of different second electrocardiographic signals by the master device, the second electrocardiographic signals also carry device identifiers of the slave devices respectively. For example, serial numbers are used to identify different electrocardiograph acquisition devices.

In the embodiment of the present disclosure, the acquisition time of the electrocardiographic voltage may be recorded by taking the startup time of the electrocardiograph acquisition device as a reference. The startup time of the first electrocardiograph acquisition device is earlier than the startup time of the second electrocardiograph acquisition device. That is, during electrocardiograph acquisition, the first electrocardiograph acquisition device is started firstly, and then the second electrocardiograph acquisition device is started. At this time, there is a time difference between the startup time of the first electrocardiograph acquisition device and the startup time of the second electrocardiograph acquisition device. According to the time difference, the second acquisition time and the third acquisition time, whether the first acquisition time and the second acquisition time are the same may be determined.

That is, the control circuit 103 is configured to determine whether the first acquisition time and the second acquisition time are the same based on the second time difference between the startup time of the first electrocardiograph acquisition device and the startup time of the second electrocardiograph acquisition device, the second acquisition time and the third acquisition time.

Exemplarily, the control circuit 103 adds the second time difference to the third acquisition time to obtain the first acquisition time, and then compares whether the first acquisition time and the second acquisition time are the same. In the case that the first acquisition time and the second acquisition time are different, the control circuit 103 subtracts the first acquisition time from the second acquisition time to obtain the first time difference. In the case that the first time difference is positive, it means that the master device starts acquiring firstly, and in the case that the first time difference is negative, it means that the slave device starts acquiring firstly.

In this system, usually, the master device (the first electrocardiograph acquisition device) is started firstly. Then the slave device (the second electrocardiograph acquisition device) is started, and sends an instruction to the master device when being started. At this time, the master device records the startup time of the slave device, such as the third second after the master device is started. The master device records a time when acquiring the second electrocardiographic voltage, i.e., the second acquisition time, such as the fifth second after the master device is started. The slave device records a time, i.e., the third acquisition time, such as 2.001th second after the slave device is started, based on the standard of the slave device when acquiring the first electrocardiographic voltage, wherein the time is converted into the time of the master device, i.e., the first acquisition time, which is the 5.001th second after the master device is started. At this time, the first acquisition time and the second acquisition time are different, and the time difference between the first acquisition time and the second acquisition time is 0.001 second.

In the embodiment of the present disclosure, the wireless transmission circuit 100 is further configured to receive an instruction from the second electrocardiograph acquisition device at the startup time.

The control circuit 103 is configured to determine the second time difference between the startup time of the first electrocardiograph acquisition device and the startup time of the second electrocardiograph acquisition device based on the instruction.

After the second time different is determined, the control circuit 103 is further configured to record the second time difference for later use.

The recording of the second time difference between the startup time of the first electrocardiograph acquisition device and the startup time of the second electrocardiograph acquisition device when the instruction is received makes a preparation for subsequent calculation of a time difference between the second acquisition time and the third acquisition time. It should be noted that since the slave device and the master device are very close to each other and both are disposed on the human body, a transmission delay may be ignored and a receiving time is taken as a startup time of the slave device.

Exemplarily, the control circuit 103 is further configured to obtain the electrocardiograph index signal by calculating the differential signal between the second electrocardiographic voltage and the first electrocardiographic voltage when the first acquisition time and the second acquisition time are the same.

In the field of electrocardiograph acquisition, usually, the differential signal of the signals detected by different electrocardiograph electrodes is used as an index. Since the directly used electrocardiographic voltage is small, amplification can be achieved by differentiation. Thus, the signal magnitude meets the demands of subsequent signal processing. In addition, the charge trend of the human body may be represented by the differential signal to meet demands of the electrocardiograph field on the signal.

In the scenario where more than two second electrocardiograph acquisition devices are configured, the control circuit can receive a plurality of first electrocardiographic signals, that is, the control circuit may obtain a plurality of first electrocardiographic voltages. When calculating the differential signals, the control circuit may differentiate the plurality of first electrocardiographic voltages and the second electrocardiographic voltage in pairs to obtain a plurality of differential signals as the electrocardiograph index signals. For example, when the control circuit obtains one first electrocardiographic voltage, and two second electrocardiographic voltages which are second electrocardiographic voltage 1# and second electrocardiographic voltage 2# according to identifiers of the slave devices, the control circuit sequentially calculates a differential signal between the first electrocardiographic voltage and the second electrocardiographic voltage 1#, a differential signal between the first electrocardiographic voltage and the second electrocardiographic voltage 2#, and a differential signal between the second electrocardiographic voltage 1# and the second electrocardiographic voltage 2#.

The first electrocardiographic voltage and the second electrocardiographic voltage are digital signals. The differential signal may be calculated in the following two ways.

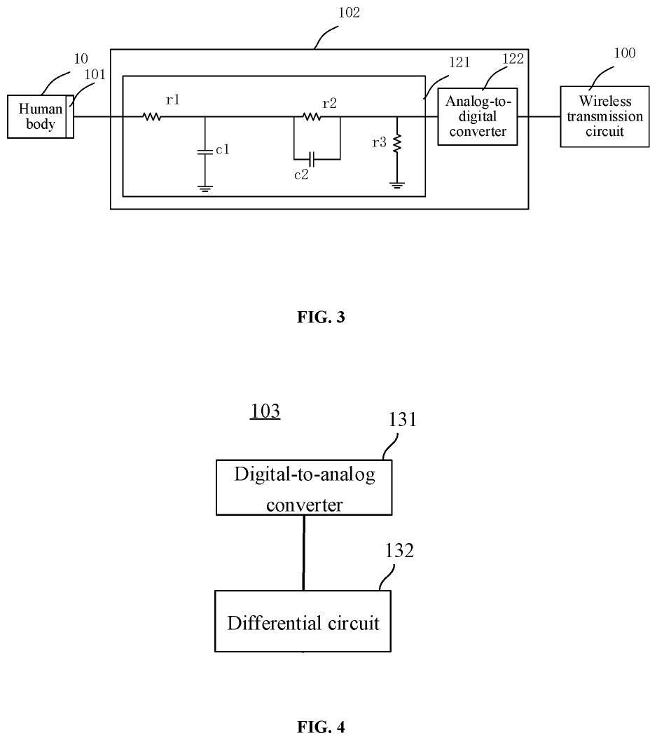

The first way is to convert the digital signal into an analog signal, and then calculate the differential signal. This way is described with reference to FIG. 4 below.

FIG. 4 is a structural block diagram of a control circuit according to the embodiment of the present disclosure. With reference to FIG. 4, the control circuit 103 may include:

a digital-to-analog converter 131, configured to perform digital-to-analog conversion on the first electrocardiographic voltage and the second electrocardiographic voltage; and

a differential circuit 132, configured to obtain a differential signal by differentially amplifying the first electrocardiographic voltage and the second electrocardiographic voltage after digital-to-analog conversion.

In this scenario, the differential signal is calculated by an analog circuit to ensure the quality of the differential signal. Since both the first electrocardiographic voltage and the second electrocardiographic voltage are digital signals, when the differential signal is calculated by the analog circuit, the first electrocardiographic voltage and the second electrocardiographic voltage need to perform digital-to-analog conversion firstly and then the differential signal is calculated by the differential circuit.

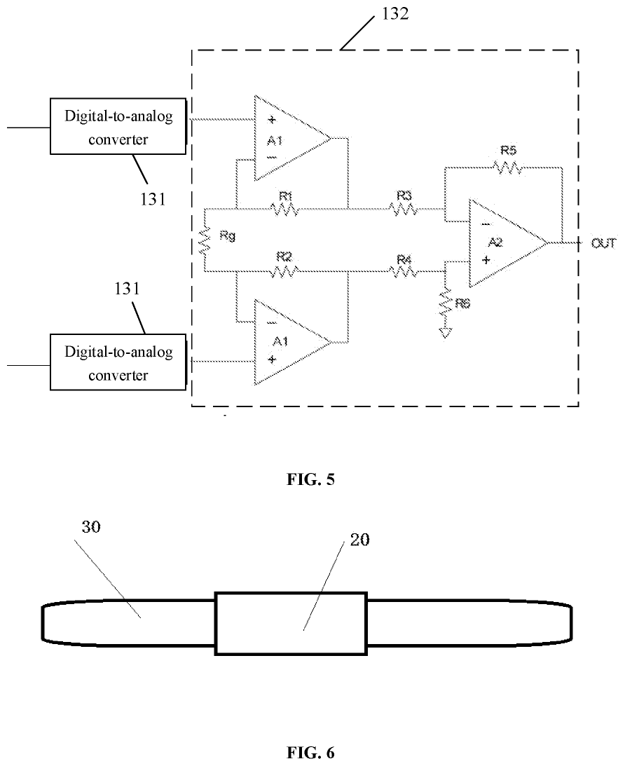

FIG. 5 is a circuit diagram of a control circuit according to the embodiment of the present disclosure. With reference to FIG. 5, the digital-to-analog converter 131 may be two digital-to-analog converters 131 or one digital-to-analog converter with two channels. In the acquisition circuit in FIG. 3, since the front-end circuit 121 is only provided with one output, the above analog-to-digital converter 122 only needs a single channel.

With reference to FIG. 5, the differential circuit 132 may include two first-stage differential amplifiers A1 and one second-stage differential amplifier A2. Non-inverting input terminals of the two first-stage differential amplifiers A1 are connected to an output terminal of the acquisition circuit 102 and an output terminal of the wireless transmission circuit 100 respectively by the digital-to-analog converters 131; inverting input terminals of the two first-stage differential amplifiers A1 are both connected to one reference electrode Rg; and the output terminals of the two first-stage differential amplifiers A1 are connected to a non-inverting input terminal and an inverting input terminal of the second-stage differential amplifier A2 respectively.

In this scenario, the differential circuit may be a second-stage differential amplifier circuit to ensure the quality of the differential signal.

As shown in FIG. 5, the differential circuit 132 may further include resistors R1 and R2 connected between the output terminals and the inverting input terminals of the two first-stage differential amplifiers A1 respectively; resistors R3 and R4 connected between the output terminals of the two first-stage differential amplifiers A1 and the two input terminals of the second-stage differential amplifier A2 respectively; a resistors R5 connected between the output terminal and the inverting input terminal of the second-stage differential amplifier A2; and a resistor R6 connected between the non-inverting input terminal of the second-stage differential amplifier A2 and the ground. The resistors R3 is connected between the output terminal of one differential amplifier A1 and the inverting input terminal of the second-stage differential amplifier A2, and the R4 is connected between the output terminal of the other differential amplifier A1 and the non-inverting input terminal of the second-stage differential amplifier A2.

The second way is to directly calculate the differential signal by a digital signal:

The second electrocardiographic voltage is directly subtracted from the first electrocardiographic voltage to obtain the differential signal. This way is simpler in circuit than the first way.

No matter which calculation way is used, it needs to ensure the synchronization of the first electrocardiographic voltage and the second electrocardiographic voltage, that is, signals which are differentiated are acquired at the same time.

Optionally, the control circuit 103 is further configured to obtain a common mode signal by averaging the second electrocardiographic voltage and the first electrocardiographic voltage, and send the common mode signal to a device with a drive circuit by the wireless transmission circuit 100. Exemplarily, the device with the drive circuit may be worn on the right leg of the human body, and the drive circuit is a right leg drive circuit. Of course, the above exemplary description does not constitute a limitation to the present disclosure. The device with the drive circuit may also be worn on other parts of the human body, such as the left leg.

The electrocardiograph acquisition circuit of the master device may calculate the common mode signal by combining electrocardiographic signals from various slave devices, and send the common mode signal to the drive circuit to suppress interference and reduce power frequency interference which is usually 50 Hz.

In the embodiment of the present disclosure, the control circuit is further configured to determine the user's heart rate based on the differential signal calculated above. Since the heartbeat of a person is periodic, a current signal generated by the periodic heartbeat and attached to the biological skin is also periodic. Since an acquisition cycle of the electrocardiograph acquisition device is much shorter than the heartbeat cycle of the person, the amplitude of the first electrocardiographic voltage acquired above changes periodically, the amplitude of the second electrocardiographic voltage also changes periodically, and the differential signal determined according to the first electrocardiographic voltage and the second electrocardiographic voltage also changes periodically. The heart rate may be determined according to a change cycle of the differential signal. When there is a plurality of differential signals, each differential signal may be used to calculate the heart rate separately, and finally the average value is taken.

In the embodiment of the present disclosure, the control circuit 103 and the wireless transmission circuit 100 may be integrated on the same chip. For example, the control circuit 103 and the wireless transmission circuit 100 both are integrated on a wireless transmission chip. The wireless transmission chip includes a master control module, which may be multiplexed as the control circuit 103, thereby saving the chip or circuit resources.

In the second electrocardiograph acquisition device, the electrocardiograph electrode 200 is configured to detect the electrical signal on biological skin.

The acquisition circuit 201 is configured to obtain the first electrocardiographic voltage by acquiring the electrical signal detected by the electrocardiograph electrode 200.

The control circuit 203 is configured to generate the first electrocardiographic signal based on the first electrocardiographic voltage obtained by the acquisition circuit 201. The first electrocardiographic signal includes the first electrocardiographic voltage and the third acquisition time of the first electrocardiographic voltage.

The wireless transmission circuit 202 is configured to send the first electrocardiographic signal under the control of the control circuit 203.

In this electrocardiograph acquisition circuit, the signal is detected by the electrocardiograph electrode, then the signal is acquired and processed by the acquisition circuit and the control circuit, and finally the processed signal is sent out by the wireless transmission circuit. Since the signal between the electrocardiograph acquisition devices is transmitted wirelessly, there is no need to arrange a connection line on the electrocardiograph acquisition device. Thus, the comfortability for the user to wear the electrocardiograph acquisition device is improved and hence the user can move freely. In addition, the wireless transmission circuit sends the acquisition time of the first electrocardiographic voltage when sending the first electrocardiographic voltage, such that the master device can determine the electrocardiographic voltages acquired at the same time according to the acquisition time, thereby ensuring the synchronization of signals of various electrocardiograph acquisition devices and avoiding reduction in signal acquisition quality caused by the wireless transmission.

The electrocardiograph acquisition circuit according to FIG. 2 is the electrocardiograph acquisition circuit of the slave device, which is provided with the same structural circuit as the master device according to FIG. 1, except that the data processing process and the control process are different.

In the embodiment of the present disclosure, the electrocardiograph acquisition circuit of the slave device not only needs to acquire the electrocardiographic signal and transmit the electrocardiographic signal to the master device, but also needs to complete synchronization with the master device.

Exemplarily, the wireless transmission circuit 202 is further configured to receive a first time difference from the first electrocardiograph acquisition device.

The control circuit 203 is further configured to output a first clock signal to the acquisition circuit 201 so as to control the acquisition of the acquisition circuit 201. The phase of the first clock signal based on the first time difference received by the wireless transmission circuit 202 is adjusted to enable the first clock signal to be synchronized with a second clock signal; and then output the first clock signal with the adjusted phase to the acquisition circuit 201.

In this scenario, the electrocardiograph acquisition circuit, as the electrocardiograph acquisition circuit of the slave device, adjusts the clock signal by receiving the first time difference from the master device to achieve synchronization.

Exemplarily, since the frequencies of the clock signals output by the control circuits of the master device and the slave device are the same, the synchronization of the clock signals can be achieved by adjusting the phase by obtaining the first time difference. Since the acquisition is performed under the control of the clock signals, the synchronization of the clock signals can cause the synchronization of the data acquisition times.

Exemplarily, the control circuit 203 calculates a phase value corresponding to the first time difference, and shifts the phase of the first clock signal according to the phase value.

For example, the control circuit 203 divides the first time difference by the cycle of the first clock signal to obtain the above phase value. The first time difference may be positive or negative which means that the shift directions are different when the phase of the first clock signal is adjusted. For example, in the case that the first time difference is positive, it means that the master device starts acquiring firstly and it needs to shift the phase of the first clock signal forwards; and in the case that the first time difference is negative, it means that the slave device starts acquiring firstly and it needs to shift the phase of the first clock signal backwards.

In addition, in order to complete the synchronization of the slave device and the master device, the wireless transmission circuit 202 of the slave device is further configured to send an instruction to the master device when the slave device is started, the instruction being configured to indicate the startup time of the slave device. The master device makes a preparation for the subsequent calculation of the first time difference by recording the second time difference between the startup time of the master device and the startup time of the slave device when receiving the instruction. For information about how the master device uses this instruction, reference can be made to the description of the control circuit in the master device above. It should be noted that since the slave device and the master device are very close to each other and are all disposed on the human body, a transmission delay may be ignored and a receiving time is taken as a startup time of the slave device.

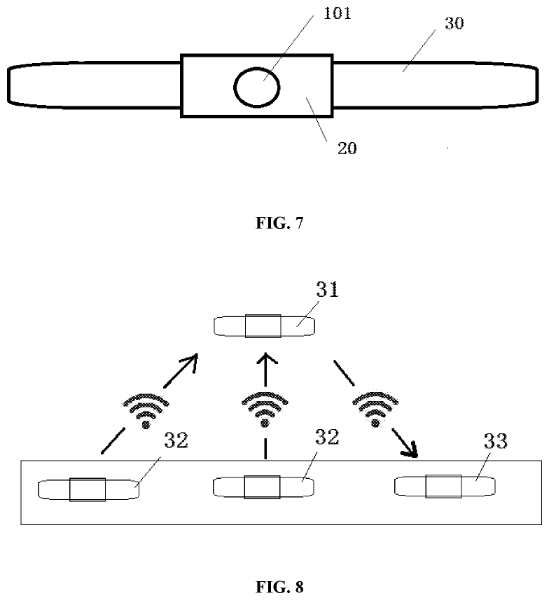

FIG. 6 and FIG. 7 are structural diagrams of an electrocardiograph acquisition terminal in two opposite directions respectively according to an embodiment of the present disclosure. With reference to FIGS. 6 and 7, the electrocardiograph acquisition terminal includes a housing 20 and a wristband 30 connected to the housing 20. The electrocardiograph electrode 101 (or the electrocardiograph electrode 200) is embedded on a surface of the housing 20, such that it may be in contact with the human body and then detect the electrocardiographic signal by the biological skin.

The electrocardiograph acquisition terminal may be a first electrocardiograph acquisition terminal or a second electrocardiograph acquisition terminal. The electrocardiograph acquisition terminal may include the electrocardiograph acquisition circuit as shown in FIG. 1 or FIG. 2.

The wireless transmission circuit 100 (or the wireless transmission circuit 202), the acquisition circuit 102 (or the acquisition circuit 201) and the control circuit 103 (or the control circuit 203) are all disposed in the housing 20, which are not shown in FIGS. 6 and 7. The electrocardiograph acquisition device may be worn on the user's limbs by adopting the wristband structure.

In addition to the above circuit, the housing 20 further includes a power source, such as a battery, to power the above circuit so as to ensure the operation of the electrocardiograph acquisition terminal.

As shown in FIGS. 6 and 7, the wristband 30 is connected to two ends of the housing 20, and two ends of the wristband 30 can be connected cooperatively, for example, connected by a buckle or a magic tape, such that the electrocardiograph acquisition terminal is detachably disposed on the limbs of the human body. The electrocardiograph acquisition terminal is bound by the wristband 30, which solves the problem of uncomfortability caused by wearing a sticky electrocardiograph electrode during electrocardiograph detection. The wristband here is not limited to being worn on the wrist of the human body. For example, it may be worn on the neck, waist or other parts, and may also be used as a waistband.

Optionally, the wireless transmission circuit 100 (or wireless transmission circuit 202) in the electrocardiograph acquisition device may also send one or more of the first electrocardiographic signal, the second electrocardiographic signal, the differential signal, and the heart rate to a separate host terminal, such as a computer device, for displaying, storing or using these data on the host terminal.

Optionally, the electrocardiograph acquisition device may also include a display module to display the calculated heart rate change, as well as information such as the time.

Optionally, the electrocardiograph acquisition device may also include a storage module for storing data such as the first electrocardiographic signal and the second electrocardiographic signal above. The storage module may be a secure digital memory (SD) card.

FIG. 8 is a structural diagram of an electrocardiograph acquisition system according to an embodiment of the present disclosure. With reference to FIG. 8, the electrocardiograph acquisition system includes a master device 31 and a slave device 32. The master device 31 includes the electrocardiograph acquisition circuit as shown in FIG. 1, and the slave device 32 includes the electrocardiograph acquisition circuit as shown in FIG. 2.

In this electrocardiograph acquisition system, the signal is detected by the electrocardiograph electrode, and then is acquired and processed by the acquisition circuit and the control circuit. In addition, the electrocardiographic signal is received from the second electrocardiograph acquisition device by the wireless transmission circuit. Since the signal between the electrocardiograph acquisition devices is transmitted wirelessly, there is no need to arrange a connection line on the electrocardiograph acquisition device. Thus, the comfortability for a user to wear the electrocardiograph acquisition device is improved and hence the user can move freely. In addition, the wireless transmission circuit carries the acquisition time of the electrocardiographic voltage when transmitting the electrocardiographic voltage, such that the electrocardiograph acquisition device can determine the electrocardiographic voltages acquired at the same time according to the acquisition time, thereby ensuring the synchronization of signals of various electrocardiograph acquisition devices and avoiding reduction in signal acquisition quality caused by the wireless transmission.

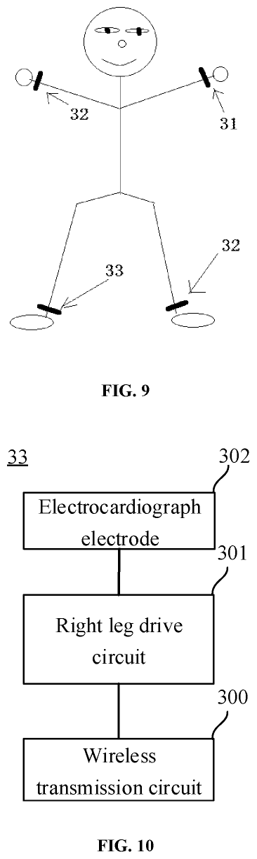

FIG. 9 is a diagram showing the position of the electrocardiograph acquisition system shown in FIG. 8. With reference to FIG. 9, the electrocardiograph acquisition system includes two slave devices 32. One master device 31 and the two slave devices 32 are disposed on two hands and one leg (such as the left leg) of a human body respectively. Thus, the split wireless electrocardiograph acquisition system may be achieved. For example, the master device 31 may be disposed on any of the two hands and the left leg.

The acquired signal that may be used to indicate the human health index is ensured by distributing the three acquisition devices on the two hands and one leg.

The two slave devices 32 send out electrocardiographic signals acquired at their positions by the wireless transmission circuit. The master device 31 may receive the electrocardiographic signals from the two slave devices 23 and then combine these electrocardiographic signals with the electrocardiographic signal acquired by itself to process the data into the electrocardiograph data of the three limb leads.

Optionally, the electrocardiograph acquisition system further includes a device 33 with a drive circuit, and the device 33 may be disposed on the right leg of the human body. By disposing the device with the drive circuit to output a common mode signal to the human body, an interference signal is eliminated and the interference suppression function is realized.

Exemplarily, the device 33 with the drive circuit may be worn on the right leg of the human body, and the drive circuit is a right leg drive circuit. Of course, the above exemplary description does not constitute a limitation to the present disclosure. The device 33 with the drive circuit may also be worn on other parts of the human body, such as the left leg.

FIG. 10 is a structural diagram of the device 33 with the drive circuit according to the embodiment of the present disclosure. With reference to FIG. 10, the device 33 includes a wireless transmission circuit 300, a drive circuit 301 and an electrocardiograph electrode 302.

The wireless transmission circuit 300 is configured to receive a common mode signal from the master device 31.

The drive circuit 301 is configured to generate a current signal by processing the common mode signal received by the wireless transmission circuit 300.

The electrocardiograph electrode 302 is configured to output the current signal generated by the drive circuit 301 to the biological skin.

The device with the drive circuit is implemented by adopting the structure similar to the electrocardiograph acquisition device, which facilitates design and manufacturing.

The drive circuit 301 includes a processing circuit, a digital-to-analog converter and a converting circuit. The processing circuit is configured to preform phase processing, such as phase shift, on the common mode signal, wherein the phase shift refers to shift the phase of the signal, such that the waveform of the signal changes, for example, a sinusoidal signal becomes a cosine signal. The digital-to-analog converter is configured to convert the common mode signal from a digital signal into an analog signal. The converting circuit is configured to convert the analog signal from a voltage signal into a current signal and outputs the current signal to the electrocardiograph electrode. The electrocardiograph electrode outputs this current signal to the biological skin.

In this electrocardiograph acquisition system, the wireless transmission mode of the master device and the slave device may use the Bluetooth wireless mesh ad hoc network technology, or Wi-Fi Mesh and other one-to-many low-power data transmission modes.

The one-to-many transmission mode means that one wireless transmission circuit receives signals from a plurality of emission sources at the same time. Since the acquisition time of the electrocardiographic signal is carried when the electrocardiographic signal is sent, the synchronization of the various signals may be realized, thereby ensuring the time consistency during differentiation processing.

Optionally, the system may further include a host terminal. The wireless transmission circuit 100 in the electrocardiograph acquisition device may also send one or more of the first electrocardiographic signal, the second electrocardiographic signal, the differential signal, and the heart rate to this host terminal for displaying, storing or using these data on the host terminal.

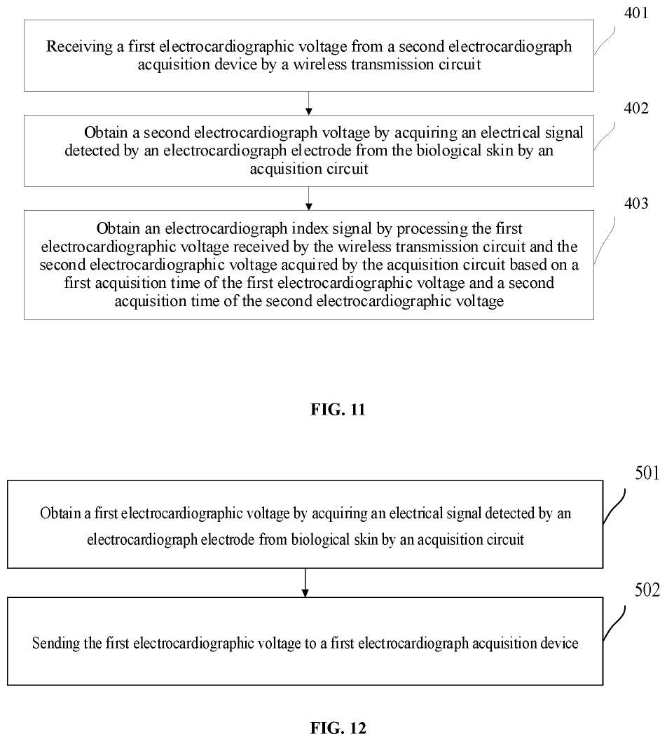

FIG. 11 is a flow chart of an electrocardiograph acquisition method according to an embodiment of the present disclosure. This method is executable by the control circuit in the electrocardiograph acquisition circuit shown in FIG. 1. With reference to FIG. 11, the method includes the following steps.

In step 401, a first electrocardiographic voltage is received from a second electrocardiograph acquisition device by a wireless transmission circuit.

The detailed process of this step may make reference to the description of the above wireless transmission circuit 100.

In step 402, a second electrocardiographic voltage is obtained by acquiring an electrical signal detected by an electrocardiograph electrode from biological skin by an acquisition circuit to obtain.

The detailed process of this step may make reference to the description of the above acquisition circuit 102.

In step 403, an electrocardiograph index signal is obtained by processing the first electrocardiographic voltage received by the wireless transmission circuit and the second electrocardiographic voltage acquired by the acquisition circuit based on a first acquisition time of the first electrocardiographic voltage and a second acquisition time of the second electrocardiographic voltage.

The detailed process of this step may make reference to the description of the above control circuit 103.

In this electrocardiograph acquisition method, the signal is detected by the electrocardiograph electrode, and then the signal is acquired and processed by the acquisition circuit and the control circuit. In addition, the electrocardiographic voltage is received from the second electrocardiograph acquisition device by the wireless transmission circuit. Since the signal between the electrocardiograph acquisition devices is transmitted wirelessly, there is no need to arrange a connection line on the electrocardiograph acquisition device. Thus, the comfortability for a user to wear the electrocardiograph acquisition device is improved and hence the user can move freely.

FIG. 12 is a flow chart of an electrocardiograph acquisition method according to an embodiment of the present disclosure. This method is executable by the control circuit in the electrocardiograph acquisition circuit shown in FIG. 2. With reference to FIG. 12, the method includes the following steps.

In step 501, a first electrocardiographic voltage is obtained by acquiring an electrical signal detected by an electrocardiograph electrode from biological skin by an acquisition circuit.

The detailed process of this step may make reference to the description of the above acquisition circuit 201.

In step 502, the first electrocardiographic voltage is sent to a first electrocardiograph acquisition device.

The detailed process of this step may make reference to the description of the above wireless transmission circuit 202.

In this electrocardiograph acquisition method, the signal is detected by the electrocardiograph electrode and then the signal is acquired and processed by the acquisition circuit and the control circuit, and finally the processed signal is sent out by the wireless transmission circuit. Since the signal between the electrocardiograph acquisition devices is transmitted wirelessly, there is no need to arrange a connection line on the electrocardiograph acquisition device. Thus, the comfortability for a user to wear the electrocardiograph acquisition device is improved and hence the user can move freely.

The above description is only alternative embodiments of the present disclosure, and is not intended to limit the present disclosure. Any modifications, equivalent replacements, improvements and the like made within the spirit and principles of the present disclosure should be included within the scope of protection of the present disclosure.

What is claimed is:

1. An electrocardiograph acquisition circuit applicable to a first electrocardiograph acquisition device, comprising:
a wireless transmission circuit, configured to receive a first electrocardiographic voltage from a second electrocardiograph acquisition device;
an electrocardiograph electrode, configured to detect an electrical signal on biological skin;
an acquisition circuit, configured to obtain a second electrocardiographic voltage by acquiring the electrical signal detected by the electrocardiograph electrode ; and
a control circuit, configured to obtain an electrocardiograph index signal by processing the first electrocardiographic voltage received by the wireless transmission circuit and the second electrocardiographic voltage acquired by the acquisition circuit based on a first acquisition time of the first electrocardiographic voltage and a second acquisition time of the second electrocardiographic voltage.

2. The electrocardiograph acquisition circuit according to claim 1, wherein the first acquisition time and the second acquisition time are times measured from a same initial time.

3. The electrocardiograph acquisition circuit according to claim 2, wherein the initial time is a startup time of the first electrocardiograph acquisition device or a startup time of the second electrocardiograph acquisition device.

4. The electrocardiograph acquisition circuit according to claim 1, wherein the control circuit is further configured to calculate a first time difference based on the first acquisition time and the second acquisition time when the first acquisition time and the second acquisition time are different, and send the first time difference to the second electrocardiograph acquisition device by the wireless transmission circuit.

5. The electrocardiograph acquisition circuit according to claim 1, wherein the wireless transmission circuit is further configured to receive the first acquisition time of the first electrocardiographic voltage from the second electrocardiograph acquisition device.

6. The electrocardiograph acquisition circuit according to claim 1, wherein the wireless transmission circuit is further configured to receive a third acquisition time from the second electrocardiograph acquisition device, wherein the third acquisition time is a time measured by taking the startup time of the second acquisition device as an initial time, and
the control circuit is configured to determine whether the first acquisition time and the second acquisition time are the same based on a second time difference between the startup time of the first electrocardiograph acquisition device and the startup time of the second electrocardiograph acquisition device, the second acquisition time and the third acquisition time.

7. The electrocardiograph acquisition circuit according to claim 6, wherein the wireless transmission circuit is further configured to receive an instruction from the second electrocardiograph acquisition device at the startup time; and
the control circuit is configured to determine the second time difference between the startup time of the first electrocardiograph acquisition device and the startup time of the second electrocardiograph acquisition device based on the instruction.

8. The electrocardiograph acquisition circuit according to claim 5, wherein the control circuit is further configured to obtain the electrocardiograph index signal by calculating a differential signal between the second electrocardiographic voltage and the first electrocardiographic voltage when the first acquisition time and the second acquisition time are the same.

9. The electrocardiograph acquisition circuit according to claim 8, wherein the control circuit is further configured to obtain a common mode signal by averaging the second electrocardiographic voltage and the first electrocardiographic voltage, and send the common mode signal to a device with a drive circuit by the wireless transmission circuit.

10. An electrocardiograph acquisition circuit applicable to a second electrocardiograph acquisition device, comprising:
an electrocardiograph electrode, configured to detect an electrical signal on biological skin;
an acquisition circuit, configured to obtain a first electrocardiographic voltage by acquiring the electrical signal detected by the electrocardiograph electrode; and
a wireless transmission circuit, configured to send the first electrocardiographic voltage to a first electrocardiograph acquisition device.

11. The electrocardiograph acquisition circuit according to claim 10, wherein the wireless transmission circuit is further configured to send a first acquisition time of the first electrocardiographic voltage, wherein the first acquisition time and a second acquisition time are times measured from a same initial time, and the second acquisition time is a time when the first electrocardiograph acquisition device acquires a second electrocardiographic voltage.

12. The electrocardiograph acquisition circuit according to claim 10, wherein the wireless transmission circuit is further configured to receive a first time difference from the first electrocardiograph acquisition device; and
the electrocardiograph acquisition circuit further comprises: a control circuit, configured to output a first clock signal, wherein a phase of the first clock signal based on the first time difference received by the wireless transmission circuit is adjusted to enable the first clock signal to be synchronized with a second clock signal, wherein the second clock signal is a clock signal of the first electrocardiograph acquisition device, and the first clock signal has a frequency the same as that of the second clock signal.

13. An electrocardiograph acquisition device, comprising: an electrocardiograph acquisition circuit, a housing, and a wristband connected to the housing, wherein the electrocardiograph acquisition circuit is the electrocardiograph acquisition circuit as defined in claim 1;
the electrocardiograph electrode is embedded on a surface of the housing; and
the wireless transmission circuit, the acquisition circuit, and the control circuit are disposed in the housing.

14. An electrocardiograph acquisition system, comprising: a master device and a slave device, wherein the slave device is a second electrocardiograph acquisition device comprising the electrocardiograph acquisition circuit as defined in claim 10, and the master device is a first electrocardiograph acquisition device comprising the electrocardiograph acquisition circuit that comprises:
a wireless transmission circuit, configured to receive a first electrocardiographic voltage from a second electrocardiograph acquisition device;
an electrocardiograph electrode, configured to detect an electrical signal on biological skin;
an acquisition circuit, configured to obtain a second electrocardiographic voltage by acquiring the electrical signal detected by the electrocardiograph electrode; and
a control circuit, configured to obtain an electrocardiograph index signal by processing the first electrocardiographic voltage received by the wireless transmission circuit and the second electrocardiographic voltage acquired by the acquisition circuit based on a first acquisition time of the first electrocardiographic voltage and a second acquisition time of the second electrocardiographic voltage.

15. The electrocardiograph acquisition system according to claim 14, comprising two slave devices, wherein the master device and the two slave devices are placed on two hands and one leg of a human body respectively.

16. The electrocardiograph acquisition system according to claim 15, further comprising a device with a drive circuit.

17. The electrocardiograph acquisition system according to claim 16, wherein the device with the drive circuit comprises:
a wireless transmission circuit, configured to receive a common mode signal from the master device;
a drive circuit, configured to generate a current signal by processing the common mode signal received by the wireless transmission circuit; and
an electrocardiograph electrode, configured to output the current signal generated by the drive circuit to the biological skin.

18. An electrocardiograph acquisition method applicable to the electrocardiograph acquisition circuit as defined in claim 1, comprising:
receiving a first electrocardiographic voltage from a second electrocardiograph acquisition device by the wireless transmission circuit;
obtaining a second electrocardiographic voltage by acquiring an electrical signal detected by the electrocardiograph electrode from the biological skin by the acquisition circuit; and
obtaining an electrocardiograph index signal by processing the first electrocardiographic voltage received by the wireless transmission circuit and the second electrocardiographic voltage acquired by the acquisition circuit based on a first acquisition time of the first electrocardiographic voltage and a second acquisition time of the second electrocardiographic voltage.

19. An electrocardiograph acquisition method applicable to the electrocardiograph acquisition circuit as defined in claim 10, comprising:
obtaining a first electrocardiographic voltage by acquiring an electrical signal detected by the electrocardiograph electrode from the biological skin by the acquisition circuit; and
sending the first electrocardiographic voltage to a first electrocardiograph acquisition device.

20. The electrocardiograph acquisition circuit according to claim 10, the wireless transmission circuit is further configured to send a third acquisition time of the first electrocardiographic voltage, wherein the third acquisition time is a time measured by taking a startup time of the second acquisition device as an initial time.
